(19) United States
(12) Patent Application Publication
(10) Pub. No.: US 2007/0078424 A1
(43) Pub. Date: Apr. 5, 2007

(54) Curved absorbent article

(76) Inventors: Lanying Zhen Wu, Paoli, PA (US); Sharon Eaton Black, Bethlehem, PA (US)

Correspondence Address: GOSZ AND PARTNERS, LLP, 450 BEDFORD STREET, LEXINGTON, MA, 02420, US

(21) Appl. No.: 11/241,779

(22) Filed: Sep. 30, 2005

(51) Int. Cl. A61F 13/15

(52) U.S. Cl. 604/385.101; 604/385.01

(57) ABSTRACT

An absorbent article is curved in a saucer like shape in the transverse direction. The absorbent article offers not only a better fitting and feeling product to the wearer, but also has better flow characteristics. The article is formed by having an absorbent core that is made from multiple strips of absorbent material arranged in a stepwise manner so as to form a gradient. These strips may be of uniform or varying width and maybe made from a combination of one or more suitable material types.

### BACKGROUND OF THE INVENTION

The present invention relates to absorbent articles that are used for containing the bodily exudate. The invention is specifically relevant to disposable absorbent articles worn by women, such as sanitary napkins, absorbing pads, pantiliner, and the like. More particularly, the present invention discloses an absorbent article that has a curvilinear shape in the transverse direction resulting in an improved leak protection mechanism.

Absorbent articles, such as, sanitary napkins, pantiliners, and feminine hygiene products, and the like are, primarily, used during the woman's menstrual period to contain menses and other vaginal discharges so as to maintain hygiene and cleanliness as well as protect garments from soiling. These absorbing articles are generally small in size, absorbent and comfortable to wear.

Absorbent articles are usually flat and configured so that they have a longitudinal dimension and transverse dimension, the latter being smaller to fit between the wearer's legs in the crotch region. Absorbing articles are intended to be positioned near the crotch region of the user's undergarment and comprise an absorbent core, a liquid pervious top sheet that touches the body of the wearer, and a liquid impervious back sheet located on the garment-facing side that acts as a fluid barrier so as to protect the user's garments from staining.

One of the persistent problems with absorbent articles of these types is that they sometimes cause hindrance in free movement of the wearer as the bottom side of the crotch is slightly curved and because the articles are usually flat in nature. Several modifications have been performed in order to make the sanitary napkin or pantiliner a better fit to the wearer. For example, articles have been made in which the edges conform to the contours of the body. However, these articles do not take into consideration the rounded portion of the body between the legs.

Furthermore, flat articles would have a tendency for higher leakage when quick absorption does not take place. Very rapid absorption is not the ultimate solution to this problem because rapid absorption may also have associated drawbacks, i.e. gel blocking. Therefore it is desirable to have a curve/saucer shaped article in which the released liquid stays in the middle portion of the article.

One modification in sanitary napkins was the introduction of flaps in the central crotch region extending from each longitudinal edge as in U.S. Pat. No. 4,589,876. The body of each flap contains a flexible axis about which the flap can fold on itself. The sanitary napkin preferentially bends at the line of juncture of each flap and the longitudinal edge of the central absorbent pad. When the sanitary napkin is in a panty for use, each flap bends upwardly at the line of juncture of the flap and longitudinal edge of the central absorbent pad to form the base of a wall. The wall extends to the flexible axis in the body of the flap, which is folded around the edge of the crotch portion of the panty.

Another more common problem experienced in existing sanitary napkins or the like is that of leakage from the crotch region. Leakage from sanitary napkins is generally attributed to a high concentration of fluid at the point where the menses exits the body and immediately contacts the surface of the napkin. At the point of deposit, the napkins absorbent material quickly becomes super-saturated. The exudate migrates radially from that point and leaks from the sides nearest the wearer's legs. This often results in the smearing of blood on the body and soiling of the undergarments. Attempts to eliminate leakage include: construction of a densified edge to hold the fluid back as in U.S. Pat. No. 4,820,295; barrier sheets surrounding the article like in U.S. Pat. No. 4,666,439, “winged” side edges that wrap around the panties; and providing sanitary napkins with elasticized longitudinal side edges. U.S. Pat. No. 4,655,789 describes the introduction of embossed channel adjacent to the longitudinal edges of crotch region. The embossed channels are activated during use by the thighs, allowing the sides of the napkin to fold upwards forming an occlusive container.

Elastic materials have been introduced to provide a curved shape for better fit and hence reduce side leakage in sanitary napkins. Examples of such use of elastic materials can be found in U.S. Pat. No. 4,701,177; U.S. Pat. No. 4,758,241; U.S. Pat. No. 4,770,657; U.S. Pat. No. 4,944,735; and U.S. Pat. No. 5,032,121. However, the application of elastic members in a sanitary napkin to achieve a curved shape is dependent on the thickness of the sanitary napkin. Also, elastic members create wrinkles along the upper part of the sanitary napkin. Moreover, the use of elastic members adds significant thickness to the product's longitudinal side barriers often resulting in a sanitary napkin that is uncomfortable to the wearer.

The respective disclosures of each of the aforementioned patents and patent publications are incorporated herein in their entirety by reference thereto.

Therefore, it is an object of the present invention to provide an absorbent article, such as a sanitary napkin, having a curved shape cross section region in the transverse direction without the application of elastic materials.

### SUMMARY OF THE INVENTION

The present invention provides non-elastic based curved shaped absorbent article, such as a sanitary napkins, pantiliners, or other such absorbent pads having a curvature in the cross or transverse direction.

The absorbing article of the present invention, in a preferred embodiment, comprises a liquid pervious top sheet, a liquid impervious back sheet, and one or more absorbent cores sandwiched between the top sheet and the back sheet. These absorbent cores are layered in a stepwise manner so as to form a saucer shaped curvature of the article. This arrangement forms a gradient in the transverse direction and a channel in the longitudinal direction.

In the preferred embodiment, the top sheet is intended to be of a narrower width as compared to back sheet. When the layers are joined together at the two longitudinal ends a curved shaped sanitary napkin is formed.

### BRIEF DESCRIPTION OF THE DRAWINGS

FIG. 1 is a top view of a typical absorbent article.

FIG. 2 is a cross sectional view of the article shown in FIG. 1.

FIG. 3 shows one embodiment of the present invention.

FIG. 4 shows an alternate embodiment of the present invention.

### DETAILED DESCRIPTION OF THE INVENTION

The present invention will now be described with reference to the accompanying drawings. The drawings and their description are merely for illustration and understanding purposes, and are in no way meant to be limiting the claimed invention.

The present invention pertains to a novel configuration of absorbent articles (such as diapers, sanitary napkins etc.) that have a curve in the transverse direction. This curve is achieved without the use of any elastic material. The absorbent core is structured and placed in such a configuration so as to cause a natural curve of the article in the desired direction.

The term “curved” or “curve” as used herein with reference to the shape of the absorbent articles, in the broadest sense, includes articles having overall shapes that represent deviations from straight lines or planar surfaces. The articles may comprise curved overall shapes that are formed by one or more curvilinear lines or surfaces, rectilinear lines or surfaces, or combinations of curvilinear and rectilinear lines or surfaces. The term “curved” is, thus, not limited solely to articles formed entirely of a curvilinear segment or segments.

The term “absorbent article”, as used herein, refers to articles that absorb and contain body exudates. More specifically, the term refers to articles that are placed against or in proximity to the body of the wearer to absorb and contain the various exudates discharged from the body. The term “absorbent article” is intended to include sanitary napkins, pantiliners, feminine hygiene products and other articles worn in the crotch region of a garment.

The term “disposable” refers to articles that are intended to be discarded after a single use.

The term “sanitary napkin”, as used herein, refers to an article, which is worn by females adjacent to the pudenda region that is intended to absorb and contain the various exudates, which are discharged from the body such as blood, menses, and urine. However, the present invention is also applicable to other feminine hygiene or cat menial pads such as pantiliners, or other absorbent articles such as incontinence pads, and the like.

The present invention, however, is not limited to the particular type or configuration of the absorbent article shown in the drawings.

The absorbent article and/or the sanitary napkin have a first end region, a second end region, and a central region between the first and second end regions, two longitudinal edges and two transverse edges. The absorbent articles comprise a liquid pervious top sheet, a liquid impervious back sheet joined to the top sheet, and an absorbent core positioned between the top sheet and the back sheet. The articles have a body contacting surface or “body surface” and a garment contacting surface region. When sanitary napkins are worn, the “body surface” is intended to be worn adjacent to the body of the wearer, while the “garment surface” is intended to be placed adjacent to the wearer's undergarments.

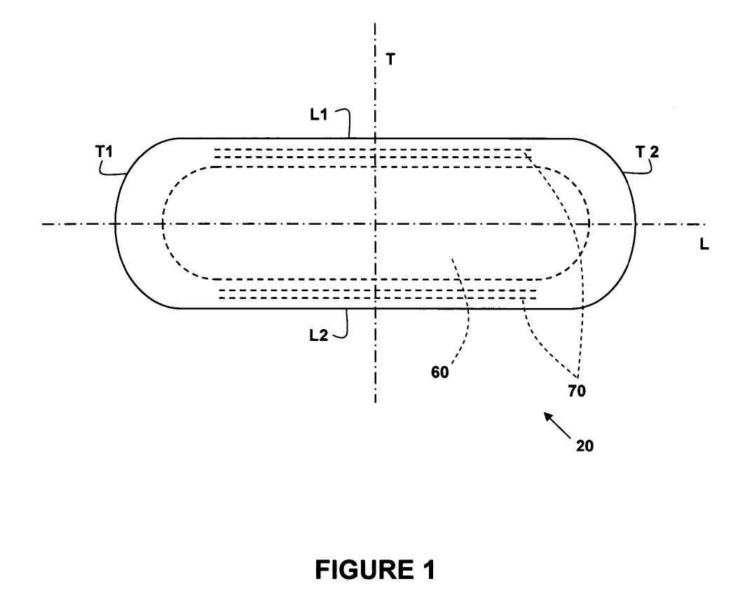

Referring to FIG. 1, which shows the top view of an absorbent article 20, the article has two centerlines, a longitudinal centerline L and a transverse centerline T, having two spaced apart longitudinal or end edges (L1, L2), and two spaced apart transverse or side edges (T1, T2), which together form the periphery of the article. The absorbent core 60 is placed in the crotch region of the article. The article 20 further comprises of a pair of leg cuffs 70 attached at the longitudinal edges in the crotch region.

The term “longitudinal”, as used herein, refers to an axis or direction in the plane of the article that is generally aligned with a vertical plane, which bisects a standing wearer into left and right body halves when the sanitary napkin is worn. The terms “transverse” or “lateral” are synonymous, and refer to the line, axis or direction generally perpendicular to the longitudinal direction, which lies within the plane of the article 20.

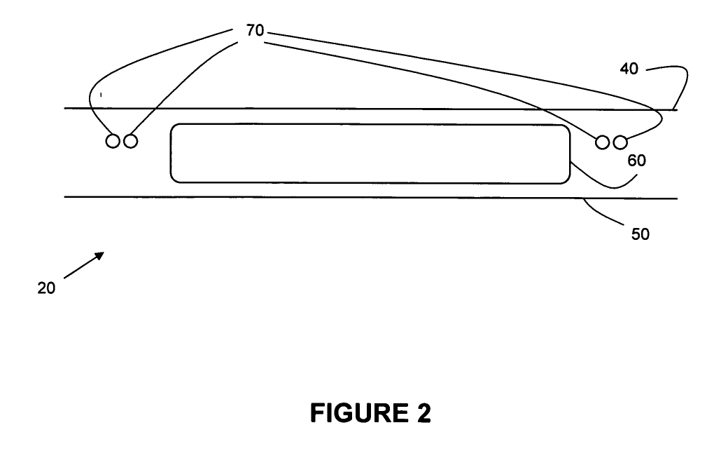

FIG. 2, is a cross sectional view of the article 20. The article preferably comprises a liquid pervious top sheet 40, a liquid impervious back sheet 50, and an absorbent core 60. The liquid permeable top sheet 40 is attached to the liquid impervious sheet 50, and the absorbent core 60 is disposed between the said top liquid permeable 40 and liquid impermeable sheet 50. Optionally, leg cuffs 70 made of strands or strips of elastic material may be disposed in the longitudinal direction. These strips make the article curve in the longitudinal direction making it conform to the shape of the body.

It is desirable to make the article curve in a saucer shape in the transverse direction so as to achieve the advantages as mentioned earlier in this document. However, adding elastic material in the transverse direction is not a sought after solution as it not only increases the bulk and the cost of the article but also adds a degree of difficulty to the manufacturing process.

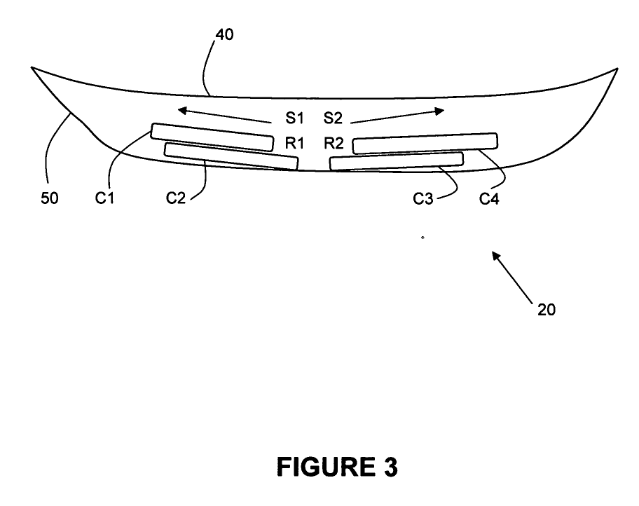

To achieve the desired curve, the present invention discloses a novel configuration, one simplistic embodiment of which is shown in FIG. 3. The absorbent material that forms the absorbent core of the article, and which is added in the cross section region of the absorbent article, is not made as one single piece. Instead multiple strips of absorbent material C1, C2, C3, C4 are arranged in a stepwise manner S1, S2 in the transverse direction. This stepwise arrangement forms a gradient in the ‘z’ direction that results in a curve in the transverse direction.

To accentuate and have a smooth finish for the curvature in the transverse direction the liquid permeable top sheet 40 is of a narrower width than the liquid impermeable backsheet 50. When these sheets are joined longitudinally they also give the article a bowl like shape in the transverse direction.

The article of the present invention can be curved by any desired amount. The article may be of relatively uniform thickness or alternatively can be profiled so that the thickness varies in any known manner. Stacking at least one relatively wide and long strip of absorbent core material sandwiched in between the liquid pervious top sheet and the liquid impervious back sheet induces thickness in the article. To smooth the angles of the curve, many thin strips of material may be placed in a stepwise manner so as to form a smoother gradient.

The stacking of superabsorbent material in the absorbent core provides a gradient in the cross section direction and channels (R1, R2) in the longitudinal direction. The superabsorbent material used in the absorbent core is of a variety known to person skilled in the art. Furthermore various strips can be made of different materials so as to customize the absorption powers of the article. For example the strips closer to the center may be made from a material that has a high absorbent capacity as well as gel-blocking characteristic.

Another customization may be such that the central portion contains strips of material that comprises a relatively low-density of superabsorbent as compared to side portions. Multiple strips of material containing the superabsorbent are stacked forming a gradient in the cross section direction.

Because of the varying stepwise arrangement of the strips and their varying width, channels R1, R2 are also formed in the longitudinal direction in the absorbent core. These channels serve to store the liquid until it is absorbed by the core thereby preventing leakage.

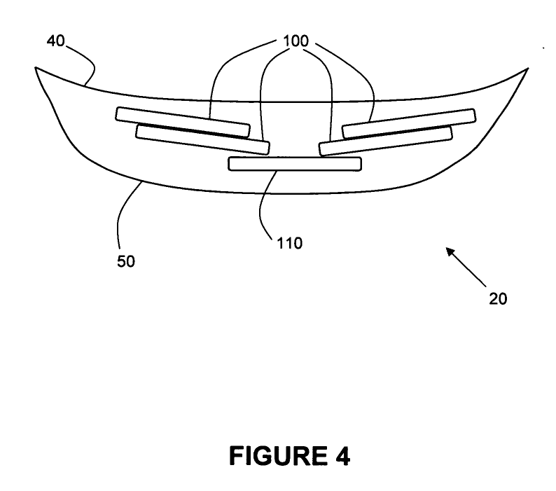

Referring to FIG. 4, this figure shows another embodiment of the invention. Strips 100, which are comprised of a relatively low density of superabsorbent uniformly distributed, are stacked over a strip 110 containing a higher density of superabsorbent material. The gradient is formed in the cross section direction, having the lower superabsorbent density strip at top, adjacent to the top sheet 40, and the higher density superabsorbent material strip adjacent to the back sheet 50.

The present invention, however, is not limited to the particular type or configuration of the absorbent article represented in the drawings. Further, such detail is used solely for the purpose of illustration of invention, and variations can be made therein by those skilled in the art without departing from the sprit and scope of the invention except as it may be limited by the claims.

1. An absorbent article comprising
a) a liquid permeable top sheet,
b) a liquid impermeable back sheet, and
c) multiple absorbent cores interposed between said liquid permeable to sheet and said liquid impermeable back sheet,
wherein said multiple absorbent cores are stacked in a stepwise gradient in the transverse direction to cause the absorbent article to curve in a saucer like shape in the transverse direction and to form open channels in the longitudinal direction extending the width of the article from the top sheet to the back sheet.

2. (canceled)

3. The absorbent article as claimed in claim 1, wherein said multiple absorbent cores are of the same density.

4. The absorbent article as claimed in claim 1, wherein said multiple absorbent cores are of different density.

5. The absorbent article as claimed in claim 1, wherein said multiple absorbent cores are of same material.

6. The absorbent article as claimed in claim 1, wherein said multiple absorbent cores are of different material.

7. The absorbent article as claimed in claim 1, wherein said multiple absorbent cores comprise airlaid material.

8. The absorbent article as claimed in claim 1, wherein the width of the topsheet is narrower than the width of the backsheet and said sheets are joined at their longitudinal edges forming a curvilinear body.

9. An absorbent article comprising:
a) a liquid permeable top sheet,
b) a liquid impermeable back sheet, and
c) multiple absorbent cores interposed between said liquid permeable to sheet and said liquid impermeable back sheet,
wherein the top sheet has a width narrower than the width of the back sheet, and said sheets are joined at their longitudinal edges forming a curvilinear body forming a saucer like shape; and
wherein said multiple absorbent cores are stacked in a stepwise gradient in the transverse direction forming open channels in the longitudinal direction extending the width of the article from the top sheet to the back sheet.

10. The absorbent article as claimed in claim 9, wherein said multiple absorbent cores are of the same density.

11. The absorbent article as claimed in claim 9, wherein said multiple absorbent cores are of different density.

12. The absorbent article as claimed in claim 9, wherein said multiple absorbent cores are of the same material.

13. The absorbent article as claimed in claim 9, wherein said multiple absorbent cores are of different material.

14. The absorbent article as claimed in claim 9, wherein said multiple absorbent cores comprise airlaid material.
